(19) United States
(12) Patent Application Publication
(10) Pub. No.: US 2015/0098973 A1
(43) Pub. Date: Apr. 9, 2015

(54) COMPOSITION FOR APPLICATION TO THE SKIN COMPRISING SILSESQUIOXANE RESIN WAX AND SOLID PARTICULATE

(71) Applicants: DOW CORNING CORPORATION, Midland, MI (US); DOW CORNING DE MÉXICO S.A. DE C.V., México City, MX (MX)

(72) Inventors: Gerald Brissette, Saginaw, MI (US); Lok Ming Eva Li, Midland, MI (US); Yihan Liu, Midland, MI (US); Mary Kay Tomalia, Midland, MI (US); Adrianna Urrutia-Gutierrez, Mexico City (MX)

(21) Appl. No.: 14/399,536

(22) PCT Filed: Apr. 29, 2013
(86) PCT No.: PCT/US2013/038584

§ 371 Date: Nov. 7, 2014

Related U.S. Application Data
(60) Provisional application No. 61/644,508, filed on May 9, 2012.

(51) Int. Cl. A61K 8/84; A61K 8/25; A61Q 19/00; A61Q 19/08; A61K 8/02; A61K 8/92

(52) U.S. Cl. CPC A61K 8/84; A61K 8/0241; A61K 8/92; A61Q 19/00; A61Q 19/08; A61K 8/25
USPC 424/401; 514/63; 424/64; 424/59; 424/63; 424/69; 514/18.8; 424/62

(57) ABSTRACT

Provided in various embodiments are compositions for topical application to the skin, comprising a silsesquioxane resin wax, at least one solid particulate with an average particle size greater than 1 micron, and one or more additional waxes.

### CROSS-REFERENCE TO RELATED APPLICATIONS

None.

### TECHNICAL FIELD

The disclosure relates to blends of waxes for topical application to the skin, methods for their preparation, and their uses in skin care compositions to provide a soft focus effect and aesthetic benefits.

### BACKGROUND

There exists a need in the market for skin care products that combine beneficial properties such as concealing skin blemishes, giving a natural look, providing good aesthetics and providing a desirable sensory feel. Some current products provide hiding power, some give a natural look, others offer a desirable sensory feel, but it has been a consistent challenge to achieve all of these attributes in a single product.

There is a wide variety of cosmetic formulations on the market that enhance the skin's appearance, but such products generally achieve that enhanced appearance by concealing the natural skin. Such products are generally opaque or semi-opaque and deposit a non-transparent layer onto the skin, thereby concealing the natural skin tone and/or color. Although there are products available that are designed to precisely match a variety of different skin tones, it is nearly impossible to precisely match each individual skin tone. As a result, under certain lighting conditions, especially under natural daylight, the presence of the cosmetic formulation may be noticeable, which is undesirable. Moreover, each individual's skin has pores and other uneven areas. Formulations that are designed to conceal the natural appearance of the skin by depositing an opaque or semi-opaque layer onto the skin tend to fill those pores and uneven areas of the skin, which is also generally visible, especially under natural light or after several hours of wear. Further, especially in warmer and more humid climates, formulations that conceal the appearance of natural skin tend to either melt and deposit themselves into the skin's uneven areas (i.e., pores, wrinkles) or get “cakey,” both of which are undesirable. There is a need in the art for products that maintain the natural look of the skin under a variety of lighting conditions.

### BRIEF SUMMARY

When silsesquioxane resin wax is combined with an appropriate solid particulate, the composition results in a unique soft focus effect which diffuses the appearance of skin blemishes and, at the same time, still allows the natural skin to be seen, moisturizes the skin, and provides a silky, velvety feel. The use of the silsesquioxane resin wax provides aesthetic benefits and comfort to the skin. The soft focus effect is synergistically enhanced due to the combination of the silsesquioxane resin wax, solid particulate, and other waxes. Thus, the present disclosure provides means to lessen the appearance of skin wrinkles, skin crow's feet, signs of intrinsic and extrinsic skin aging and benign pigment disorders, tired and/or flabby skin, age spots, fatty and/or impure skin, UV-damaged or irritated skin in a novel way—by diffusing the appearance of skin imperfections instead of covering them up.

An aspect of the present disclosure is directed to a composition comprising (a) a silsesquioxane resin wax, (b) at least one solid particulate having an average particle size greater than 1 micron, and (c) one or more additional waxes.

Another aspect of the present disclosure is directed to an anhydrous composition comprising (a) a silsesquioxane resin wax, (b) at least one solid particulate having an average particle size greater than 1 micron, (c) one or more additional waxes, and (d) one or more fluids selected from silicone fluid and organic fluid.

Another aspect of the present disclosure is directed to an emulsion composition comprising (a) a silsesquioxane resin wax, (b) at least one solid particulate having an average particle size greater than 1 micron, (c) one or more additional waxes, (d) one or more fluids selected from silicone fluid and organic fluid, (e) one or more emulsifiers, and (f) water.

Another aspect of the present disclosure is directed to a method of preparing a composition for topical application to the skin of a mammal, the method comprising the steps of mixing (a) a silsesquioxane resin wax, (b) one or more solid particulate having an average particle size greater than 1 micron, (c) one or more additional waxes, (d) optionally, one or more fluids selected from silicone fluid and organic fluid, (e) optionally, one or more emulsifiers, (f) optionally, water.

Additional aspects of the disclosure will be apparent to those of ordinary skill in the art in view of the detailed description of various embodiments provided below.

### DETAILED DESCRIPTION

Unless otherwise indicated or defined, all technical and scientific terms used herein have the same meaning as commonly understood by one of ordinary skill in the art to which the invention pertains. The terminology used herein is for describing particular embodiments only and is not intended to be limiting.

The term “ambient conditions” as used throughout the specification refers to surrounding conditions under one atmosphere of pressure, at 50 percent relative humidity, and at a temperature of from 20° C. to 25° C., unless otherwise specified.

A wax is defined as a material which is solid at ambient conditions, and which possesses a melting point. Typically, waxes used herein have a melting point ranging of from 30° C. to 130° C., alternatively of from 30° C. to 90° C.

The present disclosure relates to a composition comprising (a) a silsesquioxane resin wax, (b) at least one solid particulate having an average particle size greater than 1 micron, and (c) one or more additional waxes.

The silsesquioxane resin wax (a) is present in the composition in an amount ranging of from 10 to 70 parts, alternatively of from 30 to 60 parts, per 100 parts by weight of the total composition. The at least one solid particulate having an average particle size greater than 1 micron (b) is present in the composition in an amount ranging of from 5 to 50 parts, alternatively of from 10 to 40 parts, per 100 parts by weight of the total composition. The one or more additional waxes (c) is present in the composition in an amount ranging of from 10 to 70 parts, alternatively of from 30 to 40 parts, per 100 parts by weight of the total composition.

The ratio of the combined amount of the silsesquioxane resin wax and the one or more additional waxes to the amount of the at least one solid particulate may be in the range of from 1 to 20, alternatively of from 2 to 10.

The composition may be prepared in a form of a solid for easy transportation and storage. The solid may be in the form of a flake, a particle, a granule.

The solid is obtained by melting the waxes (a) and (c), mixing them in presence of particulate (b) and letting cool down. The solid has a melting point ranging of from 30 to 120° C., alternatively 35 to 90° C. The solid may have hardness greater than from 600 grams, as measured according to the parameters set out in Table 1 below.

TABLE 1
Hardness Measurement Parameters.
Parameters Used

| | |
|---|---|
| Probe: | 2 mm needle probe (Texture |
| | Technologies) |
| Pre-Test Speed: | 2 mm/sec |
| Test Speed: | 2 mm/sec |
| Distance: | 2 mm |
| Trigger Force: | 5 g |
| Acquisition Rate: | 200/sec |
| Hold Time: | 1 sec |
| Post-Test Speed: | 10 mm/sec |

The present disclosure also relates to an anhydrous composition for topical application to the skin comprising (a) a silsesquioxane resin wax, (b) at least one solid particulate having an average particle size greater than 1 micron, (c) one or more additional waxes, and (d) one or more fluids selected from silicone fluid and organic fluid.

The anhydrous composition may comprise the silsesquioxane resin wax (a) at a level of from 5 to 60 parts, alternatively of from 10 to 40 parts; the at least one solid particulate having an average particle size greater than 1 micron (b) at a level of from 2 to 40 parts, alternatively of from 2 to 30 parts; the one or more additional waxes (c) at a level of from 5 to 60 parts, alternatively of from 10 to 30 parts; the one or more fluids (d) at a level of from 10 to 80 parts, alternatively of from 20 to 60 parts; all per 100 parts by weight of the total anhydrous composition.

In the anhydrous composition, the ratio of the combined amount of the silsesquioxane resin wax and the one or more additional waxes to the amount of the at least one solid particulate may be ranging of from 1 to 20 and, alternatively, of from 2 to 10. The ratio of the combined amount of the silsesquioxane resin wax, solid particulate, and one or more additional waxes to the amount of fluids may be ranging of from 0.3 to 5 and, alternatively, of from 0.5 to 3.

The anhydrous composition may have hardness ranging of from 10 to 1000 grams as measured according to the parameters set out in Table 1 above, alternatively of from 40 to 600 grams, alternatively of from 175 to 600 grams.

The present disclosure also relates to an emulsion composition comprising (a) a silsesquioxane resin wax, (b) at least one solid particulate having an average particle size greater than 1 micron, (c) one or more additional waxes, (d) one or more fluids selected from silicone fluid and organic fluid, (e) one or more emulsifiers, and (f) water.

The emulsion composition may comprise the silsesquioxane resin wax (a) at a level of from 1 to 60 parts, alternatively of from 2 to 10 parts; the at least one solid particulate having an average particle size greater than 1 micron (b) at a level of from 0.5 to 40 parts, alternatively of from 0.5 to 10 parts; the one or more additional waxes (c) at a level of from 1 to 60 parts, alternatively of from 1 to 10 parts; the one or more fluids (d) at a level of from 2 to 80 parts, alternatively of from 2 to 30 parts; the one or more emulsifiers (e) at a level of from 0.1 to 20 parts, alternatively of from 1 to 10 parts; water (f) at a level of from 1 to 90, alternatively of from 40 to 80 parts; all per 100 parts by weight of the total composition.

In the emulsion composition, the ratio of the combined amount of the silsesquioxane resin wax and the one or more additional waxes to the amount of the at least one solid particulate may be ranging of from 1 to 20 or, alternatively of from 2 to 10. The ratio of the combined amount of the silsesquioxane resin wax, the solid particulate, and the one or more additional waxes to the amount of fluids may be ranging of from 0.3 to 5 or, alternatively from 0.5 to 3. The ratio of the combined amount of the silsesquioxane resin wax, the solid particulate, the one or more additional waxes, and the one or more fluids to the amount of water may be ranging of from 0.2 to 4, alternatively of from 0.3 to 1.

The emulsion composition may have a viscosity ranging of from 100 to 1,000,000 mPa/s, alternatively of from 1,000 to 100,000 mPa/s.

The present disclosure also relates to a method of preparing a composition for topical application to the skin, comprising the steps of mixing (a) a silsesquioxane resin wax, (b) one or more solid particulate having an average particle size greater than 1 micron, (c) one or more additional waxes, (d) optionally, one or more fluids selected from silicone fluid and hydrocarbon fluid, (e) optionally, one or more emulsifiers, and (f) optionally, water.

A further method of providing a soft focus effect to skin by applying to it a composition comprising (a) a silsesquioxane resin wax, (b) at least one solid particulate having an average particle size greater than 1 micron, and (c) one or more additional waxes, is also disclosed.

The composition, anhydrous composition or emulsion composition may be incorporated into any skin care or cosmetic product, such as lotions, creams, moisturizers, facial treatments, primers, concealers, correctors, pencils, foundations, make-ups, blushes, lipsticks, lip balms, eyeliners, powders and sunscreens.

### Silsesquioxane Resin Wax (a)

The incorporation of the silsesquioxane resin wax in a cosmetic or skin care formulation significantly increases the soft focus effect.

Silsesquioxane resin wax typically includes at least 40 mole % of siloxy units having the general formula (R₂R′SiO_{1/2})_{X}(R″SiO_{3/2})_{y} where x and y have a value ranging from 0.05 to 0.95, R is an alkyl group including from 1 to 8 carbon atoms, an aryl group, a carbinol group, or an amino group, R′ is a monovalent hydrocarbon having 9-40 carbon atoms, and R″ is a monovalent hydrocarbon group having 1 to 8 carbon atoms or an aryl group.

As used herein, x and y represent the mole fraction of (R₂R′SiO_{1/2}) and (R″SiO_{3/2}) siloxy units relative to each other present in the silsesquioxane resin wax. The mole fraction of siloxy units can independently vary from 0.05 to 0.95. Typically, the value of x is 0.05 to 0.95, or alternatively 0.2 to 0.8, the value of y is 0.05 to 0.95, or alternatively 0.2 to 0.8. The combination of (R₂R′SiO_{1/2}) and (R″SiO_{3/2}) siloxy units present must total at least 40 mole %, alternatively 60 mole %, or alternatively 90 mole % of all siloxy units present in the silsesquioxane resin wax.

The silsesquioxane resin wax may contain additional siloxy units such as (i) (R¹₃SiO_{1/2})ₐ, (H) (R²₂SiO_{2/2})_{b}, (iii) (R³SiO_{3/2})_{c}, or (iv) (SiO_{4/2})_{d} units, which may also be referred to as M, D, T and Q units respectively. The amount of each unit present in the silsesquioxane resin wax can be expressed as a mole fraction of the total number of moles of all siloxy units present in the silsesquioxane resin wax. Thus, the silsesquioxane resin wax may comprise the units:
(i) (R¹₃SiO_{1/2})ₐ,
(ii) (R²₂SiO_{2/2})_{b},
(iii) (R³SiO_{3/2})_{c},
(iv) (SiO_{4/2})_{d},
(v) (R₂R′SiO_{1/2})ₓ and
(vi) (R″SiO_{3/2})_{y},
wherein R, R¹, R² and R³ are independently an alkyl group having 1 to 8 carbon atoms, an aryl group, a carbinol group or an amino group; R′ is a monovalent hydrocarbon having 9-40 carbon atoms; R″ is a monovalent hydrocarbon group having 1 to 8 carbon atoms; a, b, c and d have a value of zero to 0.6; x and y have a value of 0.05 to 0.95; with the proviso that the value x+y is equal to or greater than 0.40, and the value of a+b+c+d+x+y=1.

R′ may be any linear or branched monovalent hydrocarbon having 9 to 40 carbons. Alternatively, R′ is a C18-C40 hydrocarbon group. R″ may be a methyl, ethyl, propyl, butyl, pentyl, hexyl, heptyl, octyl, or aryl group. Alternatively, R″ is propyl.

R′ and the ratio of y/x may be selected such that the silsesquioxane resin wax has a melting point greater than 30° C.

### Solid Particulate (b)

The solid particulate in the compositions imparts an increased level of light scattering, which results in an enhanced diffused or soft focus effect. While the fact that particles scatter light is a generally known phenomenon, in a skin care or cosmetic formulations, solid particles may often agglomerate in the suspending medium, resulting in an undesired effect of either reduced scattering or uneven scattering. Even if the particles do not agglomerate in the bulk formulation, once the formulation is applied onto the skin and the volatiles evaporate, particles can often agglomerate. This makes the skin appear uneven and/or unnatural or the skin may have visible and noticeable powdery residues. The combination of solid particulate with silsesquioxane resin wax reduces such agglomeration in the formulation as well as on the skin, thus providing an enhanced scattering effect.

The solid particulate may further provide for an additional effect on the skin such as sebum absorption, feel improvement, texture modification.

Examples of solid particulates include silica, alumina, mica, clay, titanium dioxide, iron oxide, zinc oxide, boron nitride, zeolite, laponite, talc, silicone resins, silicone elastomer powders, acrylic and acrylate homo- and co-polymers, nylon, polyethylene, colloidal metals, natural powders such as starch, or any combination thereof. The solid particulate may be any treated form of the previously recited examples, such as surface treatment for hydrophobization, hydrophilisation, and/or compatibilization.

Solid particulates may have an average particle size of from 100 nanometers to 100 microns, alternatively of from 1 to 50 microns, alternatively of from 2 to 20 microns.

The solid particulate may have any shape, such as for example spherical, substantially spherical, semi spherical or irregular. The solid particulate may be a solid/full, porous or hollow particulate.

### One or More Additional Waxes (c)

The one or more additional waxes may include a variety of waxes that are configured to provide one or more advantageous properties to the compositions such as compatibility with a variety of organic ingredients, melting on contact with skin, being substantive to the skin, being moisturizing, non-acnegenic, and non-comedogenic, providing a smooth and/or silky feel, adding body, and many other properties. Each of the one or more additional waxes may possess some or all of these properties.

The one or more additional waxes may be a silicone wax or an organic wax.

Examples of silicone waxes include C30-45 alkyl methicone and C30-45 olefin (MP>60° C.), Bis-PEG-18 methyl ethyl dimethyl silane, stearyl dimethicone.

Examples of organic waxes include those selected from synthetic and natural origin such as mineral waxes, animal waxes, vegetal waxes, hydrogenated oils, fatty esters and glycerides which are solid at 25° C.

Examples of organic waxes include esters derived from a monovalent saturated C16-C60 alkanol and a saturated C8-C36 monocarboxylic acid, glycerol triesters of saturated linear C18-C40 carboxylic acids, candelilla wax, carnauba wax, beeswax, saturated linear C16-C18, C20, and C22-C40 carboxylic acids, hardened castor oil, ozokerite, polyethylene wax, microcrystalline wax, ceresin, lanolin wax, rice bran wax, montan wax, orange wax, lemon wax and paraffin wax.

The one or more additional waxes is a solid at 25° C. and may have a melting point in the range of 30° C. to 130° C., alternatively of from 30° C. to 90° C.

### Fluid (d)

The fluid is a liquid at ambient conditions. Any fluid which is compatible with the silsesquioxane resin wax and which does not agglomerate the solid particulates may be used. Compatibility here refers to good mixing to achieve a blend homogeneous to the naked eye; it does not necessarily mean miscibility at a molecular level. In order to achieve soft focus, the solid particulates need to be dispersed in the fluid, and not solubilized. A particularly effective fluid is one that can simultaneously compatibilize the silsesquioxane resin wax and the additional waxes and that does not agglomerate the solid particulates, yet allows the waxes and the solid particulates to be present in the formulation as aggregates having an average size or phase domain greater than typically a few hundreds of nanometers, as observed using microscopic measurement.

The fluid may include volatile or non-volatile organic or silicone fluids or any combination(s) thereof. Volatile fluids have a boiling point below 250° C.

Examples of silicone fluid include cyclomethicones containing from 3 to 7 silicon atoms; dimethicones such as those having viscosities ranging from 0,5 to 100,000 mm²/s; organo-functional siloxanes such as caprylyl methicone, phenyl trimethicone, bis-hydroxyethoxypropyl dimethicone.

Examples of organic fluid include hydrocarbons such as isododecane, isoparaffins; esters such as myristates, carbonates; triglycerides such as capric/caprylic triglyceride; alcohols such as ethanol.

In some instances, a volatile fluid may be needed to allow the formulation to dry upon application to the skin. In other instances, a non volatile fluid with high viscosity is suitable for providing substantivity and wash off resistance. The fluid may have emollient properties.

### Emulsifier (e)

The emulsifiers suitable for use according to the present may be anionic, cationic, amphoteric and/or non-ionic.

Nonionic surfactants include dimethicone copolyols, fatty acid esters of polyols, for instance sorbitol or glyceryl mono-, di-, tri- or sesqui-oleates or stearates, glyceryl or polyethylene glycol laurates; fatty acid esters of polyethylene glycol (polyethylene glycol monostearate or monolaurate); polyoxyethylenated fatty acid esters (stearate or oleate) of sorbitol; polyoxyethylenated alkyl (lauryl, cetyl, stearyl or octyl)ethers.

Anionic surfactants include carboxylates (sodium 2-(2-hydroxyalkyloxyl)acetate)), amino acid derivatives (N-acylglutamates, N-acylgly-cinates or acylsarcosinates), alkyl sulfates, alkyl ether sulfates and oxyethylenated derivatives thereof, sulfonates, isethionates and N-acylisethionates, taurates and N-acyl N-methyltaurates, sulfosuccinates, alkylsulfoacetates, phosphates and alkyl phosphates, polypeptides, anionic derivatives of alkyl polyglycoside (acyl-D-galactoside uronate), and fatty acid soaps, and mixtures thereof.

Amphoteric and zwitterionic surfactants include betaines, N-alkylamidobetaines and derivatives thereof, proteins and derivatives thereof, glycine derivatives, sultaines, alkyl polyaminocarboxylates and alkylamphoacetates, and mixtures thereof.

### Additional Ingredients

The compositions according to the present disclosure can further include additional ingredients known in the formulation of skin care formulations. These additional ingredients may be incorporated into the compositions at levels appropriate for a particular skin care application as determined by one of ordinary skill in the art.

Additional ingredients include humectants, emollients, exfoliate agents, vitamins and their derivatives, natural extracts and their derivatives, anti-oxidants, radical scavengers, anti-acne agents, anti-inflammatory agents, antimicrobial agents, antibacterial agents, antifungal agents, anti-itch agents, peptides, proteins, sunscreens, sunfilters, skin lightening agents, tanning agents, drugs, pigments, dyes, electrolytes, anhydrous liquids, thickeners/rheology modifiers, preservatives and fragrances.

Examples of humectants include hyaluronic acid, chondroitin sulfate, pyrrolidone carboxylic acid salts, polyoxyethylene methylglucoside, polyoxypropylene methylglucoside, glycerin, propylene glycol, 1,3-butylene glycol, polyethylene glycol, sodium d,1-pyrrolidonecarbonate, sodium lactate, sorbitol, sodium hyaluronate.

Examples of emollients include non-volatile silicones, such as silicone gums, silicone resins and silicone elastomer gels.

Examples of anhydrous liquids are those that are anhydrous but water compatible, and include alcohols, polyethylene glycols, dihydric alcohols, trihydric alcohols, lower aliphatic polyhydric polyols, sugar alcohols.

Examples of thickeners/rheology modifiers include cellulose and its derivatives, bentonite, silicon dioxide, polyacrylic acids, carbomer.

A method of preparing a composition for topical application to the skin comprises the steps of mixing (a) a silsesquioxane resin wax, (b) one or more solid particulate having an average particle size greater than 1 micron, (c) one or more additional waxes, (d) optionally, one or more fluids selected from silicone fluid and hydrocarbon fluid, (e) optionally, one or more emulsifiers, and (f) optionally, water.

The mixing is carried out using conventional mixing means. Heating may be useful to melt the waxes before or during mixing.

### Soft Focus

The term “soft focus” is used here interchangeably with the words “blurry” or “fuzzy” or the like, to describe an optical effect that results in the object being seen by the observing eyes without sharp resolution. Thus, a cosmetic or skin care formulation which provides soft focus effect will, upon application to the skin, result in the natural skin being seen and the undesirable features such as wrinkles, pores, pigment spots, etc. being de-focused and not seen with sharp resolution. Therefore, the skin appears, to the naked eye, smoother, younger, and more even, while still looking natural.

The soft focus effect differs from opaque effect or appearance in that the latter, through application of an opaque skin formulation, blocks the light from reaching the skin so the natural skin, along with all its blemishes and imperfections, is not being seen. In other words, when a formulation configured to deliver an opaque effect is applied to the skin, natural skin is not seen by the naked eye; rather, the opaque formulation applied to the skin is what is seen by the naked eye. With soft focus, the light still reaches the skin and radiates back to the observing eye, however, the light is diffused, or, in optical terminology, scattered, which results in the natural skin being seen. Thus, a skin formulation that results in an opaque film on the skin covers the natural skin to make it appear more even, but often unnatural, whereas a soft focus skin formulation according to the present disclosure diffuses the light to diminish the appearance of skin imperfections, while still allowing the natural skin to be seen.

One of the ways to quantitatively assess such a soft focus effect is through measurement of light that is transmitted or reflected at a single specific angle versus scattered in different directions. A film of a sample material under consideration is first coated onto a glass slide by means of a bar applicator with a fixed gap. The gap distance determines the film thickness. This amount is representative of the usual amount of skin care formulation that one would apply to the skin. The film is then dried under ambient conditions to allow any volatile content to evaporate. If the sample material is a solid mixture, the mixture may first be melted and the molten mixture may then be coated onto the glass slide. In some cases, the glass slide as well as the bar applicator may be pre-heated in order to keep the material in a molten state during the coating process. Alternatively, the solid mixture may first be dispersed in a liquid carrier or made into an emulsion and the dispersion or the emulsion be coated accordingly.

The glass slide with the coating is then placed in a spectrophotometer were an incident light is then shined through the sample and the total transmittance, diffuse transmittance, total reflectance, and diffuse reflectance are measured. The instrument is capable of measurement within a range of wavelengths (˜800-200 nm) corresponding to the visible and UV region.

An incident light (IL) is shined through a glass slide (GS) coated with a sample composition (SC). The incident light is partially transmitted, partially reflected and partially absorbed. The total transmittance (TT) is the transmitted light collected within the forward half of the sphere. Diffuse transmittance (DT) is total transmittance minus the light collected within a forward solid angle of 8°. The total reflectance (TR) is the reflected light collected within the backward half of the sphere. Diffuse reflectance (DR) is total reflectance minus the light collected with a reflected angle equal to the incident angle (DR=TR−SR). Light reflected at an angle equal to the incident angle is called specular reflection (SR). These quantities are typically expressed as a percentage of the incident light beam intensity. The amount of light absorbed (AB) is measured by subtracting the combined total transmittance (TT) and total reflectance (TR) from 100%. Thus, AB=100%−(TT+TR).

For a sample material to exhibit the property of soft focus, the amount of incident light that is absorbed by the sample material is below 25%, alternatively below 10%.

A desired soft focus effect of the compositions according to the present disclosure is one where both DT and DR are as high as possible with most of the light being transmitted instead of reflected or absorbed. When a large amount of light is absorbed by the composition, the skin will appear dim or dark, instead of radiant, which is undesirable. When most of the incident light is reflected off of the composition, the skin may appear bright but the reflection is from the composition rather than from the skin, so the natural skin is not being seen. A desirable effect is one that has a minimal amount of absorption by the composition and a relatively low amount of total reflection. A higher fraction of the diffused light from the light that is transmitted through or reflected from the composition leads to a blurrier effect; i.e., the skin appears more even. In other words, higher DT/TT and DR/TR ratios are more desirable. A typical optical effect according to the present invention is when DT/TT is greater than 0.6, alternatively greater than 0.7, and DR/TR is greater than 0.8, alternatively greater than 0.85, TT/TR is at least 2.5, alternatively at least 5, and the ratio of AB/(TT+TR+AB) is no higher than 0.25, alternatively no higher than 0.1. All the preceding measurements are based upon measurement on a film of 37 microns thickness made of the compositions. It is to be understood that a different film thickness may yield different measurements for the above parameters; for instance, the thicker the film, the lower the TT and the higher the AB. One skilled in the art should adjust the above criteria appropriately to target a desirable soft focus effect.

A soft focus effect may be provided to the skin by applying to it a composition comprising (a) a silsesquioxane resin wax, (b) at least one solid particulate having an average particle size greater than 1 micron, and (c) one or more additional waxes.

Compositions prepared according to the embodiments of the present disclosure can be used in various personal care, cosmetic, and medical applications. The composition, anhydrous composition or emulsion composition may be present in skin care or cosmetic products within a form selected from lotions, creams, moisturizers, facial treatments, primers, concealers, correctors, pencils, foundations, make-ups, blushes, lipsticks, lip balms, eyeliners, powders, and sunscreens.

### EXAMPLES

These examples are intended to illustrate the invention to one of ordinary skill in the art and should not be interpreted as limiting the scope of the invention as set forth in the claims. All parts and percentages in the examples are on a weight basis and all measurements were indicated at 25° C., unless indicated to the contrary. All mixings of the components were carried out with bench top mixers using propeller type of blades, unless indicated to the contrary.

### Example 1

Solid composition 1: 16.15 grams of silica, 48.01 grams of silsesquioxane resin wax, 18.01 grams of C18-36 acid triglyceride, and 18.03 grams of a 3:1 mixture of stearyl dimethicone and octadecene were combined together, heated at 85-90° C. under stirring until homogeneous, then let cool down to room temperature. The solid was in the form of a flake of 1-5 mm thickness.

### Example 2

Solid composition 2: 25.81 grams of silica, 38.41 grams of silsesquioxane resin wax, 18.06 grams of C18-36 acid triglyceride, and 18.16 grams of a 3:1 mixture of stearyl dimethicone and octadecene were combined together, heated at 85-90° C. under stirring until homogeneous, then let cool down to room temperature. The solid was in the form of a flake of 1-5 mm thickness.

### Example 3

Solid composition 3: 16 grams of a mixture of HDI trimethylol hexyllactone crosspolymer and silica, 48 grams of silsesquioxane resin wax, 18 grams of stearic acid and 18 grams of cetearyl alcohol were combined together, heated at 85-90° C. under stirring until homogeneous, then let cool down to room temperature. The solid was in the form of a flake of 1-5 mm thickness.

### Examples 4 to 13

The anhydrous compositions containing the ingredients listed in Table 2 were prepared as follows:
Combine the fluids (d) in an appropriate sized beaker, mix until homogeneous
Add the waxes (a) and (c) to the fluids (d), heat to melt the waxes and mix until homogeneous
Add the solid particulates (b) until all particulates are wetted and the mixture is homogeneous
Let cool down to room temperature under constant stirring

TABLE 2

| | | | | | | | | | | |
|---|---|---|---|---|---|---|---|---|---|---|
| | Exam- | Exam- | Exam- | Exam- | Exam- | Exam- | Exam- | Exam- | Exam- | Exam- |
| INCI | ple 4 | ple 5 | ple 6 | ple 7 | ple 8 | ple 9 | ple 10 | ple 11 | ple 12 | ple 13 |

Silsesquioxane resin wax (a)

| | | | | | | | | | | |
|---|---|---|---|---|---|---|---|---|---|---|
| C 30-45Alkyldimethylsilyl | 23.81 | 14.29 | 24.60 | 21.74 | 15.15 | 12.82 | 24.53 | 8.00 | 8.00 | 8.30 |
| Polypropylsilsesquioxane (1) | | | | | | | | | | |

Solid particulate (b)

| | | | | | | | | | | |
|---|---|---|---|---|---|---|---|---|---|---|
| Ethylene/Acrylic Acid Copolymer (2) | | | | | | | | 2.00 | 2.00 | |
| Silica (3) | 6.67 | 11.43 | 4.90 | 17.39 | 12.12 | 20.51 | 4.90 | | | |
| Silica silylate (1) | 0.95 | | 0.80 | | | | 0.79 | | | 0.50 |
| CI 77891 (and) Triethoxycaprylylsilane (4) | | | | | | | 0.10 | | | |
| Mica, CI 77891, Triethoxycaprylylsilane (4) | | | | | | | 0.01 | | | |
| Mica, CI 77491, CI 77891, | | | | | | | 0.01 | | | |
| Triethoxycaprylylsilane (4) | | | | | | | | | | |
| Dimethicone/Vinyl Dimethicone Crosspolymer | | | | | | 10.26 | | 2.00 | 2.00 | |
| (and) Silica (1) | | | | | | | | | | |

Additional wax (c)

| | | | | | | | |
|---|---|---|---|---|---|---|---|
| Stearyl Dimethicone (and) Octadecene (1) | 9.52 | 5.71 | 9.80 | 8.70 | 6.06 | 5.13 | 9.81 |
| Hydrogenated Soy Polyglycerides (and) C15-23 | 9.52 | | | | | | |
| Alkane (1) | | | | | | | |
| Hydrogenated Soybean Oil (and) Hydrogenated | 1.90 | | | | | | |
| Soy Polyglycerides (and) C15-23 Alkane (1) | | | | | | | |
| Tribehenin (5) | | 5.71 | | | 3.03 | | |
| C18-36 Acid Triglyceride (5) | | | | 8.70 | 3.03 | | 9.81 |
| Ozokerite (6) | | | 9.80 | | | | |

Fluid (d)

| | | | | | | | | | | |
|---|---|---|---|---|---|---|---|---|---|---|
| Caprylic/Capric Triglyceride (5) | | 5.71 | | | | | | | | |
| Dimethicone 1.5 mm²/s (and) Dimethicone | | | | | | | | 55.00 | | |
| Crosspolymer (1) | | | | | | | | | | |
| Dimethicone 2 mm²/s (and) Dimethicone | | | | | | | | | 55.00 | |
| Crosspolymsr (1) | | | | | | | | | | |
| Cyclopentasiloxane (and) Dimethicone | | | | | | | | | | 63.90 |
| Crosspolymer (and) Dimethicone/Vinyl | | | | | | | | | | |
| Dimethicone Crosspolymer (and) Dimethiconol (1) | | | | | | | | | | |
| Dimethicone 2 mm²/s (1) | 47.62 | 57.14 | 50.10 | 43.48 | 60.61 | 41.03 | 50.04 | | | |
| Caprylyl Methicone (1) | | | | | | 10.26 | | 31.00 | 31.00 | 27.30 |
| C11-13 Isoparaffin (and) Dimethiconol (and) | | | | | | | | 2.00 | 2.00 | |
| Isohexadecane (and) Dimethicone (1) | | | | | | | | | | |
| TOTAL (parts) | 99.99 | 99.99 | 100.00 | 100.01 | 100.00 | 100.01 | 100.00 | 100.00 | 100.00 | 100.00 |

### Examples 14 to 25

The emulsion compositions using the ingredients listed in Table 3 were prepared as follows:
Phase A: xanthan gum was dispersed in water with disodium EDTA until homogeneous, then the mixture was heated to 80° C., and PEG 100 Stearate (and) Glyceryl Stearate and Sorbitan Stearate were added and mixed until molten and homogeneous
Phase B: fluids were combined with waxes, and heated up to 80° C., under mixing until homogeneous, then particulates were added with mixing until all particulates were wetted and the mixture was homogeneous
Phase B was poured slowly into phase A under sufficient stirring to allow emulsification; then heating was removed; subsequently phase C was added. When temperature was below 45° C., phase D was added; mixing was pursued for an additional 15 minutes. The emulsion was homogenized using a high shear mixing device for 1 minute and allowed to cool to room temperature.

### Example 26

The emulsion composition using the ingredients listed in Table 3 was prepared as follows:
xanthan gum was dispersed in water with disodium EDTA until homogeneous, then the mixture was heated to 80° C., and PEG 100 Stearate (and) Glyceryl Stearate and Sorbitan Stearate were added and mixed until molten and homogeneous,
next, dimethicone 2 mm²/s and caprylic/capric triglyceride were added to the mixture and mixed until homogeneous,
the solid composition, in the form of flakes, of Example 3 was then added to the emulsion and mixed until molten and homogeneous, and sufficient stirring was maintained to allow emulsification
heating was then removed and phase C was added,
when temperature was below 45° C., phase D was added
mixing was pursued for an additional 15 minutes.

TABLE 3

| | | | | | | | |
|---|---|---|---|---|---|---|---|
| | Exam- | Exam- | Exam- | Exam- | Exam- | Exam- | Exam- |
| INCI | ple 14 | ple 15 | ple 16 | ple 17 | ple 18 | ple 19 | ple 20 |

Phase A

| | | | | | | | |
|---|---|---|---|---|---|---|---|
| Water | 61.86 | 61.86 | 51.35 | 50.35 | 50.35 | 64.24 | 66.85 |
| Disodium EDTA (7) | 0.05 | 0.05 | 0.05 | 0.05 | 0.05 | 0.05 | 0.05 |
| Xanthan Gum (8) | 0.40 | 0.40 | 0.40 | 0.40 | 0.40 | 0.40 | 0.40 |
| PEG 100 stearate (and) Glyceryl | 0.75 | 0.75 | 0.75 | 0.75 | 0.75 | 0.75 | 0.75 |
| Stearate (5) | | | | | | | |
| Sorbitan Stearate (5) | 1.75 | 1.75 | 1.75 | 1.75 | 1.75 | 1.75 | 1.75 |

Phase B

| | | | | | | | |
|---|---|---|---|---|---|---|---|
| Dimethicone 2 mm²/s (1) | 17.17 | 17.17 | | | | 10.71 | 8.00 |
| Caprylyl Methicone (1) | | | 20.00 | 20.00 | 16.00 | 2.68 | |
| Caprylic/Capric Triglyceride (5) | 2.00 | 2.00 | | | | | 4.00 |
| Dicaprylyl Carbonate (9) | | | | | | | |
| Myreth-3 Myristate (9) | | | | | | | |
| Stearic acid | | | | | | | |
| Cetearyl alcohol | | | | | | | |
| Dimethicone 1.5 mm²/s (and) | | | 2.00 | 10.00 | 10.00 | | |
| Dimethicone Crosspolymer (1) | | | | | | | |
| C 30-45Alkyldimethylsilyl | 3.59 | 3.59 | 4.00 | 4.00 | 4.00 | 3.35 | 2.50 |
| Polypropylsilsesquioxane (1) | | | | | | | |
| Stearyl Dimethicone (and) | 1.43 | 1.43 | | | | 1.34 | |
| Octadecene (1) | | | | | | | |
| Tribehenin (5) | | 1.43 | | | | | |
| C18-36 Acid Triglyceride (5) | 1.43 | | | | | | |
| Behenyl Alcohol (9) | | | | | | | |
| Silica silylate (1) | | | | | | | |
| Dimethicone/Vinyl Dimethicone | | | 7.00 | | | 2.68 | 4.00 |
| Crosspolymer (and) Silica (1) | | | | | | | |
| HDI trimethylol hexyllactone | | | | | | | |
| crosspolymer (and) silica (2) | | | | | | | |
| Silica (2) | | | | | | | |
| Silica (3) | 2.87 | 2.87 | | | | 5.35 | |
| CI 77891 (and) | | | | | | | |
| Triethoxycaprylylsilane (4) | | | | | | | |
| Mica, CI 77891, | | | | | | | |
| Triethoxycaprylylsilane (4) | | | | | | | |
| Mica, CI 77491, CI 77891, | | | | | | | |
| Triethoxycaprylylsilane (4) | | | | | | | |

Phase C

| | | | | | | | |
|---|---|---|---|---|---|---|---|
| Glycerin (10) | 5.00 | 5.00 | 10.00 | 10.00 | 10.00 | 5.00 | 10.00 |
| Propylene Glycol (11) | 1.00 | 1.00 | 2.00 | 2.00 | 2.00 | 1.00 | 1.00 |
| Butylene Glycol (11) | | | | | | | |
| Phase D | | | | | | | |
| Dimethicone/Vinyldimethicone | | | | | | | |
| Crosspolymer (and) C12-14 | | | | | | | |
| Pareth-12 (1) | | | | | | | |
| Ethylene/Acrylic Acid Copolymer (2) | | | | | 4.00 | | |
| Glycerin, Water, Butylene Glycol, | | | | | | | |
| Carbomer, Polysorbate 20, | | | | | | | |
| Palmitoyl Oligopeptide, Palmitoyl | | | | | | | |
| Tetrapeptide-7 (12) | | | | | | | |
| Methyl Gluceth-20 (13) | | | | | | | |
| Phenoxyethanol, Methyparaben, | 0.70 | 0.70 | 0.70 | 0.70 | 0.70 | 0.70 | 0.70 |
| Butylparaben, Ethylparaben, | | | | | | | |
| Propylparaben (14) | | | | | | | |
| TOTAL (parts) | 100.00 | 100.00 | 100.00 | 100.00 | 100.00 | 100.00 | 100.00 |
| Viscosity (mPa/s) | | | | 18600 | 7750 | 24483 | |

| | | | | | | |
|---|---|---|---|---|---|---|
| | Exam- | Exam- | Exam- | Exam- | Exam- | Exam- |
| INCI | ple 21 | ple 22 | ple 23 | ple 24 | ple 25 | ple 26 |

Phase A

| | | | | | | |
|---|---|---|---|---|---|---|
| Water | 48.35 | 56.86 | 66.86 | 73.85 | 48.65 | 69.15 |
| Disodium EDTA (7) | 0.05 | 0.05 | 0.05 | 0.05 | 0.05 | 0.05 |
| Xanthan Gum (8) | 0.40 | 0.40 | 0.40 | 0.40 | 0.40 | 0.40 |
| PEG 100 stearate (and) Glyceryl | 0.75 | 0.75 | 0.75 | 0.75 | 0.75 | 0.75 |
| Stearate (5) | | | | | | |
| Sorbitan Stearate (5) | 1.75 | 1.75 | 1.75 | 1.75 | 1.75 | 1.75 |

Phase B

| | | | | | | |
|---|---|---|---|---|---|---|
| Dimethicone 2 mm²/s (1) | | 17.17 | 17.17 | 8.26 | 10.00 | 10.00 |
| Caprylyl Methicone (1) | 20.00 | | | | 9.60 | |
| Caprylic/Capric Triglyceride (5) | | 2.00 | 2.00 | | 3.00 | 1.20 |
| Dicaprylyl Carbonate (9) | | | | | 3.00 | |
| Myreth-3 Myristate (9) | | | | | 0.50 | |
| Stearic acid | | | | | | 1.80 |
| Cetearyl alcohol | | | | | | 1.80 |
| Dimethicone 1.5 mm²/s (and) | | | | | | |
| Dimethicone Crosspolymer (1) | | | | | | |
| C 30-45Alkyldimethylsilyl | 4.00 | 3.59 | 3.59 | 4.05 | 3.00 | 4.80 |
| Polypropylsilsesquioxane (1) | | | | | | |
| Stearyl Dimethicone (and) | | 1.43 | 1.43 | 1.62 | | |
| Octadecene (1) | | | | | | |
| Tribehenin (5) | | | | | | |
| C18-36 Acid Triglyceride (5) | | 1.43 | 1.43 | 1.62 | 0.50 | |
| Behenyl Alcohol (9) | | | | | 0.50 | |
| Silica silylate (1) | | | | 0.13 | 0.10 | |
| Dimethicone/Vinyl Dimethicone | | | | | | |
| Crosspolymer (and) Silica (1) | | | | | | |
| HDI trimethylol hexyllactone | | | | | | 1.60 |
| crosspolymer (and) silica (2) | | | | | | |
| Silica (2) | | | | | 4.00 | |
| Silica (3) | | 2.87 | 2.87 | 0.81 | | |
| CI 77891 (and) | | | | 0.02 | | |
| Triethoxycaprylylsilane (4) | | | | | | |
| Mica, CI 77891, | | | | 0.002 | | |
| Triethoxycaprylylsilane (4) | | | | | | |
| Mica, CI 77491, CI 77891, | | | | 0.002 | | |
| Triethoxycaprylylsilane (4) | | | | | | |

Phase C

| | | | | | | |
|---|---|---|---|---|---|---|
| Glycerin (10) | 10.00 | 10.00 | | 5.00 | 6.00 | 5.00 |
| Propylene Glycol (11) | 2.00 | 1.00 | 1.00 | 1.00 | 1.00 | 1.00 |
| Butylene Glycol (11) | | | | | 2.00 | |
| Phase D | | | | | | |
| Dimethicone/Vinyldimethicone | 12.00 | | | | | |
| Crosspolymer (and) C12-14 | | | | | | |
| Pareth-12 (1) | | | | | | |
| Ethylene/Acrylic Acid Copolymer (2) | | | | | | |
| Glycerin, Water, Butylene Glycol, | | | | | 4.00 | |
| Carbomer, Polysorbate 20, | | | | | | |
| Palmitoyl Oligopeptide, Palmitoyl | | | | | | |
| Tetrapeptide-7 (12) | | | | | | |
| Methyl Gluceth-20 (13) | | | | | 0.50 | |
| Phenoxyethanol, Methyparaben, | 0.70 | 0.70 | 0.70 | 0.70 | 0.70 | 0.70 |
| Butylparaben, Ethylparaben, | | | | | | |
| Propylparaben (14) | | | | | | |
| TOTAL (parts) | 100.00 | 100.00 | 100.00 | 100.01 | 100.00 | 100.00 |
| Viscosity (mPa/s) | 9200 | 16050 | | | | |

TABLE 4
suppliers

| | |
|---|---|
| (1) | = Dow Corning |
| (2) | = Kobo Products |
| (3) | = Presperse |
| (4) | = Sensient |
| (5) | = Croda |
| (6) | = Strahl & Pitsch |
| (7) | = AkzoNobel |
| (8) | = CP Kelco |
| (9) | = Cognis |
| (10) | = Fisher Scientific |
| (11) | = Acros |
| (12) | = Sederma |
| (13) | = Lubrizol |
| (14) | = Clariant |

Soft focus was measured as described above, on films of thickness set at 37 microns in a PerkinElmer Lambda 650 UV-Vis Spectrophotometer equipped with a 150 mm Integrating sphere, at a wavelength of 550 nm. Results are given in Table 5.

The compositions in Examples 11 to 26 resulted in absorption below 5%, in a ratio Total Transmittance/Total Reflectance above 2.5, in a ratio Diffuse Transmittance/Total Transmittance above 60% and a ratio Diffuse Reflectance/Total Reflectance above 80%.

TABLE 5

| | | | Total | Diffuse | Diffuse | |
|---|---|---|---|---|---|---|
| | | | Transmittance/ | Transmittance/ | Reflectance/ | |
| | Total | Total | Total | Total | Total | |
| | Transmittance | Reflectance | Reflectance | Transmittance | Reflectance | Absorption |
| | (%) | (%) | (%) | (%) | (%) | (%) |
| Example 11 | 89.64 | 9.11 | 9.84 | 89.04 | 96.84 | 1.25 |
| Example 12 | 89.98 | 9.06 | 9.93 | 87.50 | 96.43 | 0.96 |
| Example 13 | 84.40 | 12.43 | 6.79 | 81.13 | 95.56 | 3.17 |
| Example 14 | 89.04 | 9.56 | 9.31 | 88.51 | 97.52 | 1.40 |
| Example 15 | 86.38 | 11.35 | 7.61 | 96.12 | 99.61 | 2.27 |
| Example 16 | 91.64 | 7.81 | 11.73 | 66.49 | 85.05 | 0.55 |
| Example 17 | 90.20 | 8.43 | 10.70 | 60.13 | 83.51 | 1.37 |
| Example 18 | 88.69 | 9.31 | 9.53 | 72.87 | 90.66 | 2.00 |
| Example 19 | 90.43 | 8.63 | 10.48 | 86.83 | 96.20 | 0.94 |
| Example 20 | 92.16 | 7.27 | 12.68 | 64.00 | 81.08 | 0.57 |
| Example 21 | 92.51 | 7.70 | 12.01 | 87.11 | 94.77 | −0.21* |
| Example 22 | 89.50 | 9.16 | 9.77 | 76.72 | 92.58 | 1.34 |
| Example 23 | 89.60 | 8.70 | 10.30 | 79.24 | 94.88 | 1.70 |
| Example 24 | 91.02 | 7.92 | 11.49 | 73.20 | 87.12 | 1.06 |
| Example 25 | 90.19 | 8.52 | 10.59 | 73.08 | 90.30 | 1.29 |
| Example 26 | 88.71 | 8.28 | 10.71 | 93.21 | 97.11 | 3.00 |

*within measurement uncertainty

1. A composition, comprising, per 100 parts by weight of the total composition:
(a) a silsesquioxane resin wax;
(b) at least one solid particulate having an average particle size greater than 1 micron;
(c) one or more additional waxes;
(d) optionally, one or more fluids selected from silicone fluid and organic fluid;
(e) optionally, one or more emulsifiers; and
(f) optionally, water.

2. The composition of claim 1, comprising:
10 to 70 parts of ingredient (a);
5 to 50 parts of ingredient (b); and
10 to 70 parts of ingredient (c),
wherein the ratio of combined ingredients (a) and (c) to (b) ranges from 1 to 20.

3. (canceled)

4. The composition of claim 1, comprising:
5 to 60 parts of ingredient (a);
2 to 40 parts of ingredient (b);
5 to 60 parts of ingredient (c); and
10 to 80 parts of ingredient (d),
wherein the ratio of combined ingredients (a) and (c) to (b) ranges from 1 to 20 and the ratio of combined ingredients (a), (b) and (c) to (d) ranges of from 0.3 to 5.

5. (canceled)

6. The composition of claim 1, comprising:
1 to 60 parts of ingredient (a);
0.5 to 40 parts of ingredient (b);
1 to 60 parts of ingredient (c);
2 to 80 parts of ingredient (d);
0.1 to 20 parts of ingredient (e); and
1 to 90 parts of ingredient (f),
wherein the ratio of combined ingredients (a) and (c) to (b) ranges from 1 to 20, the ratio of the combined ingredients (a), (b) and (c) to (d) ranges of from 0.3 to 5, and the ratio of combined ingredients (a), (b), (c) and (d) to (f) ranges of from 0.2 to 4.

7. The composition of claim 1, wherein the composition is configured to provide a soft focus effect as defined by a diffused transmitted light intensity being at least 60% of a total transmitted light intensity and a diffused reflected light intensity being at least 80% of a total reflected light intensity when a light is shined through a film made of the composition having a thickness of 37 microns, whereupon the ratio of the total transmitted light intensity to the total reflected light intensity is at least 2.5 and an amount of light absorbed by the film is no more than 25% of the incident light.

8. The composition of claim 1, wherein ingredient (a) is a silsesquioxane resin wax including at least 40 mole % of siloxy units having the general formula:
(R₂R′SiO_{1/2})ₓ(R″SiO_{3/2})_{y}, (1)
wherein x and y have a value of 0.05 to 0.95; R is an alkyl group including from 1 to 8 carbon atoms, an aryl group, a carbinol group, or an amino group; R′ is a monovalent hydrocarbon including 18 to 40 carbon atoms; R″ is a monovalent hydrocarbon group including 1 to 8 carbon atoms or an aryl group; and wherein R′ and a ratio of y/x are selected such that the silsesquioxane wax has a melting point greater than 30° C.

9. The composition of claim 1, wherein ingredient (b) is selected from silica, alumina, mica, clay, titanium dioxide, iron oxide, zinc oxide, boron nitride, zeolite, laponite, talc, silicone resins, silicone elastomer powders, acrylic and acrylate homo- and co-polymers, nylon, polyethylene, colloidal metals, natural powders, starch, or any combination thereof.

10. The composition of claim 1, wherein ingredient (b) is treated.

11. The composition of claim 1, wherein ingredient (b) has an average particle size of from 1 to 50 microns.

12. The composition of claim 1, wherein ingredient (c) is solid at 25° C. and is selected from silicone wax or organic wax from natural or synthetic origin.

13. The composition according of claim 1, wherein ingredient (d) is selected from volatile or non-volatile organic or silicone fluid or combination(s) thereof.

14. The composition of claim 1, wherein the composition is present in a skin care or cosmetic product within a form selected from lotions, creams, moisturizers, facial treatments, primers, concealers, correctors, pencils, foundations, make-ups, blushes, lipsticks, lip balms, eyeliners, powders, and sunscreens.

15. The composition of claim 14, further comprising one or more skin care active ingredients selected from humectants, emollients, exfoliate agents, vitamins and their derivatives, natural extracts and their derivatives, anti-oxidants, radical scavengers, anti-acne agents, anti-inflammatory agents, antimicrobial agents, antibacterial agents, antifungal agents, anti-itch agents, peptides, proteins, sunscreens, sunfilters, skin lightening agents, tanning agents, drugs, pigments, dyes, electrolytes, anhydrous liquids, thickeners/rheology modifiers, preservatives and fragrances.

16. A method of preparing a formulation for topical application to the skin of a mammal, comprising the steps of mixing:
(a) a silsesquioxane resin wax;
(b) one or more solid particulate having an average particle size greater than 1 micron;
(c) one or more additional waxes;
(d) optionally, one or more fluids selected from silicone fluid and hydrocarbon fluid;
(e) optionally, one or more emulsifiers; and
(f) optionally, water.

17. A method of providing a soft focus effect to skin by applying to it a composition comprising:
(a) a silsesquioxane resin wax;
(b) at least one solid particulate having an average particle size greater than 1 micron;
(c) one or more additional waxes;
(d) optionally, one or more fluids selected from silicone fluid and organic fluid;
(e) optionally, one or more emulsifiers; or
(f) optionally, water.

18. The method of claim 17, wherein ingredient (a) is a silsesquioxane resin wax including at least 40 mole % of siloxy units having the general formula:
(R₂R′SiO_{1/2})ₓ(R″SiO_{3/2})_{y}, (1)
wherein x and y have a value of 0.05 to 0.95; R is an alkyl group including from 1 to 8 carbon atoms, an aryl group, a carbinol group, or an amino group; R′ is a monovalent hydrocarbon including 18 to 40 carbon atoms; R″ is a monovalent hydrocarbon group including 1 to 8 carbon atoms or an aryl group; and wherein R′ and a ratio of y/x are selected such that the silsesquioxane wax has a melting point greater than 30° C.

19. The method of claim 16, wherein ingredient (a) is a silsesquioxane resin wax including at least 40 mole % of siloxy units having the general formula:
(R₂R′SiO_{1/2})ₓ(R″SiO_{3/2})_{y}, (1)
wherein x and y have a value of 0.05 to 0.95; R is an alkyl group including from 1 to 8 carbon atoms, an aryl group, a carbinol group, or an amino group; R′ is a monovalent hydrocarbon including 18 to 40 carbon atoms; R″ is a monovalent hydrocarbon group including 1 to 8 carbon atoms or an aryl group; and wherein R′ and a ratio of y/x are selected such that the silsesquioxane wax has a melting point greater than 30° C.

20. The composition of claim 2, wherein ingredient (a) is a silsesquioxane resin wax including at least 40 mole % of siloxy units having the general formula:
(R₂R′SiO_{1/2})ₓ(R″SiO_{3/2})_{y}, (1)
wherein x and y have a value of 0.05 to 0.95; R is an alkyl group including from 1 to 8 carbon atoms, an aryl group, a carbinol group, or an amino group; R′ is a monovalent hydrocarbon including 18 to 40 carbon atoms; R″ is a monovalent hydrocarbon group including 1 to 8 carbon atoms or an aryl group; and wherein R′ and a ratio of y/x are selected such that the silsesquioxane wax has a melting point greater than 30° C.

21. The composition of claim 4, wherein ingredient (a) is a silsesquioxane resin wax including at least 40 mole % of siloxy units having the general formula:
(R₂R′SiO_{1/2})ₓ(R″SiO_{3/2})_{y}, (1)
wherein x and y have a value of 0.05 to 0.95; R is an alkyl group including from 1 to 8 carbon atoms, an aryl group, a carbinol group, or an amino group; R′ is a monovalent hydrocarbon including 18 to 40 carbon atoms; R″ is a monovalent hydrocarbon group including 1 to 8 carbon atoms or an aryl group; and wherein R′ and a ratio of y/x are selected such that the silsesquioxane wax has a melting point greater than 30° C.

22. The composition of claim 6, wherein ingredient (a) is a silsesquioxane resin wax including at least 40 mole % of siloxy units having the general formula:
(R₂R′SiO_{1/2})ₓ(R″SiO_{3/2})_{y}, (1)
wherein x and y have a value of 0.05 to 0.95; R is an alkyl group including from 1 to 8 carbon atoms, an aryl group, a carbinol group, or an amino group; R′ is a monovalent hydrocarbon including 18 to 40 carbon atoms; R″ is a monovalent hydrocarbon group including 1 to 8 carbon atoms or an aryl group; and wherein R′ and a ratio of y/x are selected such that the silsesquioxane wax has a melting point greater than 30° C.
